SEQUENCE LISTING (6 sequences)

SEQ ID NO: 1 - DNA, 20 bp, artificial sequence
  Synthetic Oligonucleotide
  primer_bind (1)..(20): Forward primer for Oct-4
atggctggacacctggcttc

SEQ ID NO: 2 - DNA, 21 bp, artificial sequence
  Synthetic Oligonucleotide
  primer_bind (1)..(21): Reverse primer for Oct-4
ccaggttctcttgtctacctc

SEQ ID NO: 3 - DNA, 22 bp, artificial sequence
  Synthetic Oligonucleotide
  primer_bind (1)..(22): Forward primer for Sca-I
ggacacttctcacactacaaag

SEQ ID NO: 4 - DNA, 23 bp, artificial sequence
  Synthetic Oligonucleotide
  primer_bind (1)..(23): Reverse primer for Sca-I
taacacagactccatcagggtag

SEQ ID NO: 5 - DNA, 20 bp, artificial sequence
  Synthetic Oligonucleotide
  primer_bind (1)..(20): Forward primer for GADPH
accacagtccatgccatcac

SEQ ID NO: 6 - DNA, 20 bp, artificial sequence
  Synthetic Oligonucleotide
  primer_bind (1)..(20): Reverse primer for GADPH
tccaccaccctgttgctgta

(19) United States
(12) Patent Application Publication
(10) Pub. No.: US 2007/0087332 A1
(43) Pub. Date: Apr. 19, 2007

(54) Pulmonary stem cells, related methods and kits of parts

(76) Inventors: John Cheng-Po Yu, La Jolla, CA (US); Alice Lin-Tsing Yu, La Jolla, CA (US); Thai-Yen Ling, Taipei (CN)

Correspondence Address: GREENBERG TRAURIG LLP, 2450 COLORADO AVENUE, SUITE 400E, SANTA MONICA, CA, 90404, US

(21) Appl. No.: 11/252,458

(22) Filed: Oct. 17, 2005

(51) Int. Cl. C12Q 1/70; C12N 7/00; C12N 5/08; C12N 15/86

(52) U.S. Cl. 435/5; 435/366; 435/235.1; 435/456; 977/802

(57) ABSTRACT

Isolated pulmonary stem cells, the isolated pulmonary stem cells being slowly dividing Oct-4 expressing cells forming individual colonies and able to undergoing terminal differentiation into a mature phenotype in vitro; a method to identify stem cells of a tissue; a kit for the identification of stem cell of a tissue; a method for identifying an infective agent able to infect pulmonary cells; a method for the production of a virus able to infect pulmonary cells and method for identifying a compound interfering with infection of a cell by an infective agent.

### FIELD OF THE DISCLOSURE

The present disclosure relates to stem cells and associated methods and kits of parts. In particular, it relates to pulmonary stem cells and associated methods and kits of parts.

### BACKGROUND

Stem cells are found in a number of tissues and organs from the earliest stages of development (embryonic stem cells) to adulthood (adult stem cells). While embryonic stem cells have been isolated and studied separately from non stem cells, adult stem cells have been identified only as a part of a larger cell population including non-stem cells.

Adult stem cells are reported to be present in various tissues, including skin, bone marrow, muscle, brain, and other tissues. Adult stem cells act as a repair system, continually replenishing ageing tissues with normal cells. This ability to create new body tissue is what makes stem cells such an exciting prospect for medical science. They offer the possibility of generating new cells to replace diseased and damaged body tissues in conditions such as Parkinson, diabetes, cancer and Alzheimer's.

The lung is comprised of bronchial and alveolar tissues, which are specialized for air conduction, and gas exchange, respectively. These tissues remodel in response to a wide variety of environmental insults and biological pathogens. Whether there exist distinct pulmonary stem cell populations that are responsible for lung remodeling and repair after injury remains a matter of debate (6).

Several groups have suggested potential candidates for pulmonary stem cells including the basal and mucous secretory cells of the trachea (7, 8), Clara cells of the bronchioles (9), and alveolar type 2 pneumocytes (10). In addition, some variant forms of Clara cells, which reside within neuroepithelial bodies or bronchoalveolar duct junctions, have been implicated in detoxification of xenobiotics and repopulation of bronchioles in lung tissues (11, 12, 13). Moreover, a few studies suggest that CD34 and Sca-I positive cells from bone marrow engrafted and differentiated into epithelial cells of multi-organs including lung tissues (14).

Lungs are target of numerous infective agents, some responsible of fatal diseases, such as severe acute respiratory syndrome-associated coronavirus (SARS-CoV). Patients SARS-CoV present with fever and pneumonia that initially responds to antiviral therapy. Viral titers decline rapidly after day ten following initial infection. Despite this decline in viral load, twenty percent of patients clinically develop acute respiratory distress syndrome by week three, which is associated with high mortality. These SARS-infected lung tissues demonstrate diffuse alveolar damage with edema, hemorrhage, and mononuclear infiltration.

Also several diseases affecting lungs are associated with acute respiratory distress syndrome (ARDS). ARDS treatments are targeted to control inflammatory response. But the severity and recovery of lung injury also depend on epithelial cell function. In fact, the predominant pathological finding is diffuse alveolar epithelial damage. The alveolar epithelium is also the site of alveolar fluid reabsorption and plays a major role in the development of lung fibrosis associated with ARDS.

### SUMMARY

According to a first aspect isolated adult stem cells are disclosed, the adult stem cells being slowly dividing cells forming individual colonies in vitro, the adult stem cell expressing marker Oct-4 the adult stem cells able to undergoing terminal differentiation into mature phenotype. In particular, the adult stem cells herein disclosed are pulmonary stem cells.

According to a second aspect a method to identify stem cells in a tissue is disclosed, the method comprising: identifying slowly dividing Oct-4 expressing cells of the tissue, the identified slowly dividing Oct-4 expressing cells able to form colonies in vitro, the identified slowly dividing Oct-4 expressing cells, upon isolation in primary culture, able to differentiate in a mature phenotype.

According to a third aspect a kit of parts for the identification of stem cell in a tissue is disclosed, the kit of parts comprising a first identifier for the identification of slowly dividing cells and a second identifier for identifying the expression of the marker Oct-4, the first and the second identifier to be used to identify slowly dividing Oct-4 expressing cells, the identified slowly dividing Oct-4 expressing cells able to form colonies in vitro and to differentiate in a mature phenotype, being stem cells.

According to a fourth aspect method for cultivating adult stem cells in vitro is disclosed, the method comprising: providing a tissue comprising tissue cells; isolating the tissue cells from the tissue; incubating the isolated tissue cells in a suitable medium; applying to the isolated incubated cell a cell sorting procedure to obtain colonies in primary cultures; and selecting the colonies in primary colonies that express marker Oct-4, the colonies able to differentiate into a mature phenotype.

According to a fifth aspect a method for identifying an infective agent able to infect pulmonary cells is disclosed, the method comprising contacting an infective agent with the above identified isolated pulmonary stem cell; detecting the level of infection of the isolated pulmonary stem cell by the infective agent infection; and comparing the detected level of infection with a predetermined threshold level, the threshold level indicative of development of an infection of the cell.

According to a sixth aspect a method for the production of an infective agent, in particular a virus, is disclosed, the method comprising: contacting a virus particle with an isolated pulmonary stem cell in a culture; and collecting virus particles from the culture. In particular, contacting a virus particle with an isolated pulmonary stem cell in a culture can be performed in a serum-free culture condition. The viral particle can be SARS, influenza, in particular influenza A, and avian flu viruses.

According to a seventh aspect a method for identifying a compound interfering with infection of a pulmonary stem cell by an infective agent, is disclosed the method comprising contacting a candidate compound with an isolated pulmonary stem cell; contacting the isolated pulmonary stem cell with a candidate infective agent; detecting the level of infection of the isolated pulmonary stem cell by the infective agent; and comparing the detected level of infection with a predetermined threshold level, the threshold level indicative of development of infection of the cell. In particular, the infective agent can be a virus and the virus can be SARS, influenza viruses, such as influenza A, and avian flu viruses.

### DRAWINGS

The patent or application file contains at least one drawing executed in color. Copies of this patent or patent application publication with color drawing(s) will be provided by the Office upon request and payment of the necessary fee.

The above-mentioned features and objects of the present disclosure will become more apparent with reference to the following description taken in conjunction with the accompanying drawings wherein like reference numerals denote like elements and in which:

FIG. 1 shows immunohistochemical staining for BrdU of formalin fixed lung tissues of neonatal mouse, immediately after five day injections; BrdU retaining cells are shown as brown stains; BrdU non-retaining cells are shown as blue stains;

FIG. 2 shows immunohistochemical staining chase for BrdU of formalin fixed lung tissues of neonatal mouse, for 4 weeks after BrdU labeling; BrdU retaining cells are shown as brown stains and are indicated by arrows; BrdU non-retaining cells are shown as blue stains;

FIG. 3 shows immunohistochemical staining 4 weeks after BrdU labeling of formalin fixed lung tissues of neonatal mouse in a serial section (5 to 10 μ in thickness) of the section showed in FIG. 2 with anti-pancytokeratin antibodies (AE1/3) to demonstrate the bronchiolar area (purple stains); and blue stains in nuclei of all cells in the tissue section;

FIG. 4 shows immunohistochemical staining of formalin fixed lung tissues of neonatal mouse with antibodies directed against Oct-4; Oct-4 expressing cells are shown as brown stains indicated by arrows; Oct-4 non-expressing cells are shown as blue stains;

FIG. 5 shows immunohistochemical staining of formalin fixed lung tissues of neonatal mouse in a serial section (5 to 10 μ in thickness) of the section showed in FIG. 4 with anti-pancytokeratin antibodies (AE1/3) to demonstrate the bronchiolar area (purple stains); and blue stains in nuclei of all cells in the tissue section;

FIG. 6 shows immunohistochemical double staining for BrdU and Oct-4 of formalin fixed lung tissues of neonatal mouse, for 4 weeks after BrdU labeling; panel A shows staining for BrdU wherein BrdU retaining cells are shown as green stains indicated by arrows; panel B shows staining for Oct-4 wherein Oct-4 expressing cells are shown as red stains indicated by arrows; panel C shows merged images of Panel A and B, wherein the stains are indicated by asterisks;

FIG. 7 shows a phase-contrast photograph of a primary culture from lung tissue of neonatal mouse; a pulmonary epithelial colony is indicated by arrow;

FIG. 8 shows a diagram reporting the results of a flow cytometric sorting of pulmonary cells isolated from lung tissues; on the x axis the relative numbers indicating the size of the cells are shown; on the y-axis the fluorescence intensity is shown. The R1 region indicates the size of cells as well as the relative fluorescence intensity after incubation of cells with 2-bromoacetamidoethyl sulfonamide, a fluorogenic supravital dye. The R1 region refers to the subpopulation of cells with high fluorescence intensity and large size of cells;

FIG. 9 shows a phase-contrast photograph of a primary culture from lung tissue of neonatal mouse after enrichment; large pulmonary epithelial colonies are indicated by arrows;

FIG. 10 shows a phase-contrast photograph of a primary pulmonary cell culture;

FIG. 11 shows immunohistochemical staining of the primary pulmonary cell culture of FIG. 10 with polyclonal antibodies anti-Oct-4; Oct-4 expressing cells are shown as red stains; panel A is an amplified image of the area defined by the white box in FIG. 11, the amplified image showing Oct-4 expression in the nuclei of the cells;

FIG. 12 shows counter staining with DAPI of the immunostained primary pulmonary cell culture of FIG. 10 merged with immunostaining pattern of FIG. 10; cell nuclei are shown as blue stains; Oct-4 expressing cells are shown as red stains;

FIG. 13 shows a phase-contrast photograph of a primary pulmonary cell culture;

FIG. 14 shows immunohistochemical staining with antibodies anti-SSEA-I; SSEA-1 expressing cells are shown as red stains;

FIG. 15 shows counter staining with DAPI of the immunostained primary pulmonary cell culture of FIG. 13 merged with immunostaining pattern of FIG. 14; cell nuclei are shown as blue stains; cytoplasm of SSEA-I expressing cells are shown as red stains; panel A is an amplified image of the area defined by the white box in FIG. 15, the amplified image showing SSEA-1 was found on the cell surface and in cytoplasm of the pulmonary colony cells shown in FIG. 15;

FIG. 16 shows a phase-contrast photograph of a primary pulmonary cell culture;

FIG. 17 shows immunohistochemical staining with antibodies with antibodies anti Sca-1; Sca-1 expressing cells are shown as red stains;

FIG. 18 shows counter staining with DAPI of the immunostained primary pulmonary cell culture of FIG. 16 merged with immunostaining pattern of FIG. 17; cell nuclei are shown as blue stains; cytoplasm of Sca-I expressing cells are shown as red stains; panel A is an amplified image of the area defined by the white box in FIG. 17, the amplified image showing Sca-1 was found on the cell surface and in cytoplasm of the pulmonary colony cells shown in FIG. 17;

FIG. 19 shows (A) phase contrast photograph and (B) the respective immunostaining merged with DAPI of primary pulmonary cultures treated with specific antibodies directed against cytokeratin-7; nuclei of all cells are shown as blue stains; cytokeratin-7 positive cells are shown as red stains;

FIG. 20 shows (A) phase contrast photograph and (B) the respective immunostaining merged with DAPI of primary pulmonary cultures treated with specific antibodies directed against Clara cell secretion protein; nuclei of all cells are shown as blue stains; Clara cell secretion protein positive cells are shown as red stains;

FIG. 21 shows (A) phase contrast photograph and (B) the respective cytochrome P450 enzyme staining; cytochrome P450 enzyme activities in the cells is shown by blue stains;

FIG. 22 shows the RT-PCR analysis for the transcription of mRNA for Oct-4 in the cells of pulmonary epithelial colonies; results of RT-PCR performed on mouse testis Sertoli cell (TM4) for negative control is shown on lane 1; results of RT-PCR performed on cells picked up from pulmonary epithelial colonies is shown on lane 2; results of RT-PCR performed on mouse embryonic germ cells for positive control is shown on lane 3; a marker for molecular weight is shown on the M lane; position of GADPH used as internal standard is indicated by an asterisk;

FIG. 23 shows the RT-PCR analysis for the transcription of mRNA for Sca-1 in the cells of pulmonary epithelial colonies; results of RT-PCR performed on VeroE6 cells for negative control is shown on lane 1; results of RT-PCR performed on cells picked up from pulmonary epithelial colonies is shown on lane 2; results of RT-PCR performed on BW5147 cells for positive control is shown on lane 3; a marker for molecular weight is shown on the M lane; position of GADPH used as internal standard is indicated by an asterisk;

FIG. 24 shows a phase-contrast photographs of the cells after subculture for day 5;

FIG. 25 shows immunohistochemical staining merged with DAPI counter-staining of the primary pulmonary cell culture of FIG. 24 with anti-surfactant protein C antibodies; cells expressing surfactant protein C are shown as red stains; the nuclei of the cells are shown as blue stains;

FIG. 26 shows a phase-contrast photograph of the cells after subculture for day 9;

FIG. 27 shows immunohistochemical staining merged with DAPI counter-staining of the primary pulmonary cell culture of FIG. 26 with anti-aquaporin-5 antibodies; cells expressing aquaporin-5 are shown as red stains; the nuclei of the cells are shown as blue stains;

FIG. 28 shows a phase contrast photographs for pulmonary epithelial cells infected with SARS-CoV for 8 h; the pulmonary epithelial colonies are indicated by an arrow;

FIG. 29 shows immunohistochemical staining of the primary pulmonary cell culture of FIG. 28 with antibodies against nucleocapside protein of SARS-CoV; positive cells are shown as red stains;

FIG. 30 shows counter staining with DAPI of the immunostained primary pulmonary cell culture of FIG. 29 merged with immunostaining pattern of FIG. 29; nuclei of all cells are shown as blue stains; SARS-CoV positive cells are shown as red stains; panel A is an amplified image of the area defined by the white box in FIG. 30, the amplified image showing that SARS-CoV was found on the cell surface and in cytoplasm of the pulmonary colony cells shown in FIG. 30;

FIG. 31 shows a phase contrast photograph for pulmonary epithelial cells infected with SARS-CoV for 24 h; the pulmonary epithelial colonies are indicated by an arrow;

FIG. 32 shows immunohistochemical staining of the primary pulmonary cell culture of FIG. 31 with antibodies against nucleocapside protein of SARS-CoV; positive cells are shown as red stains;

FIG. 33 shows counter staining with DAPI of the immunostained primary pulmonary cell culture of FIG. 32 merged with immunostaining pattern of FIG. 32, nuclei of all cells are shown as blue stains; SARS-CoV positive cells are shown as red stains; panel A is an amplified image of the area defined by the white box in FIG. 33, the amplified image showing that SARS-CoV was found on the cell surface and in cytoplasm of the pulmonary colony cells shown in FIG. 33;

FIG. 34 shows a phase contrast photograph for pulmonary epithelial cells infected with Influenza A/WSN/33 virus for 8 h; the pulmonary epithelial colonies are indicated by an arrow;

FIG. 35 shows immunohistochemical staining of the primary pulmonary cell culture of FIG. 34 with anti-influenza A virus specific antibodies; positive cells are shown as green stains;

FIG. 36 shows counter staining with DAPI of the immunostained primary pulmonary cell culture of FIG. 35 merged with immunostaining pattern of FIG. 35; nuclei of cells are shown as blue stains; influenza A positive cells are shown as green stains;

FIG. 37 shows an electron micrograph of a primary pulmonary cell culture after 16 hours post-infection of SARS-CoV; swollen Golgi vesicles in the cells cytoplasm are indicated by arrows;

FIG. 38 shows a higher magnification of the electron micrograph of FIG. 37 wherein the swollen Golgi vesicles in the cells, cytoplasm are indicated by arrows; SARS Co-V are shown as black particles inside the Golgi vesicles;

FIG. 39 shows a higher magnification of the electron micrograph of FIG. 37 wherein SARS Co-V are shown as black particles; a SARS Co-V particle with spike proteins attached to the plasma membrane to enter via coated pit mediated is shown by an arrow;

FIG. 40 shows a phase contrast photograph for pulmonary epithelial cells wherein pulmonary epithelial colonies are indicated by an arrow;

FIG. 41 shows immunohistochemical staining of the primary pulmonary cell culture of FIG. 40 with anti-ACE2 specific monoclonal antibody to characterize putative receptors for SARS-CoV in pulmonary epithelial colonies; positive cells are shown as red stains;

FIG. 42 shows counter staining with DAPI of the immunostained primary pulmonary cell culture of FIG. 41 merged with immunostaining pattern of FIG. 41; nuclei of all cells are shown as blue stains; ACE-2 positive cells are shown as red stains; panel A is an amplified image of the area defined by the white box in FIG. 42, the amplified image showing that ACE-2 was found on the cell surface and in cytoplasm of the pulmonary colony cells shown in FIG. 42;

FIG. 43 shows a diagram reporting the bioactivity of SARS-CoV particles produced from a pulmonary primary culture; on the x-axis the post-infection time where the SARS-CoV particles are produced is reported; on the y-axis the titration of the SARS-CoV particles expressed as plaque numbers in outcome of a plaque assay is reported;

FIG. 44 shows a phase contrast photography of VeroE6 cells after infection with SARS-CoV particles produced by a pulmonary primary culture; cells showing cytopathic effects formation are indicated by arrows; and

FIG. 45 shows immunostaining for SARS-CoV used with antibodies directed against the nucleocapside of SARS-CoV; positive cells are shown as red stains.

### DETAILED DESCRIPTION

According to a first aspect, isolated adult stem cells are disclosed. The phrase “stem cells” refers to primal undifferentiated cells, which retain the ability to grow or to differentiate into other cell types. In particular, the stem cells herein disclosed are a rare subpopulation of pulmonary cells with the characteristics of stem cells, identifiable in lung tissue of mammals such as mice and humans. The term “tissue” refers to a group of cells along with their associated intercellular substances, the cells, of one or several types, serving a specific function within a multicellular organism, such as a connective tissue or an epithelium.

The identified pulmonary stem cells are slow cycling cells and express Oct-4, a marker associated with embryonic stem cells. The phrase “slowly cycling cells” or “slowly dividing cells” refers to cells having a doubling time >48 h, typically slowly-dividing cells divide once in about every three to four days.

The term “marker” refers to a protein, metabolite, gene, other compound, or biological event which is indicative of a relevant biological condition of a biological material, wherein a “biological condition” is a state or state of being of or relating to biology or life and living processes, and “biological material” is any material able of self-replication under appropriate condition, such as viruses, eukaryotic or prokaryotic cells, unicellular or multicellular organism, and other material identifiable by a person skilled in the art. For marker Oct-4, the biological material is a mammalian cell and the biological condition is the state of primal undifferentiated cells retaining the ability to grow or to differentiate into other cell types, which characterize the stem cells.

Markers, typically proteins, DNA sequences, metabolites or biological events, are detectable according to procedures known to a person skilled in the art which include use of identifiers suitable to specifically detect the marker. An “identifier” is a molecule, metabolite or other compound, such as antibodies, DNA or RNA oligonucleotides, able to discover or determine the existence, presence, or fact of or otherwise detect a marker under procedures identifiable by a person skilled in the art; exemplary identifiers are primary and secondary antibodies and oligonucleotides as described in the examples, exemplary procedures are immunostaining, immunofluorescence, and RT-PCR, as described in the examples. The term “express” or “expression” refers to a process by which a marker comes into existence in a cell, for example when the marker results from a gene's coded information, “expression” refers to the process by which the information is converted into the marker, and when the marker is a biological event such as BrdU retention, the process by which the biological event is initiated.

The pulmonary stem cells can also express stem cell specific markers other than Oct-4 and/or markers associated with biological conditions such as specific cell type, cell lineages and/or cell status. The phrase “cell type” refers to a distinct morphological or functional form of a cell. The phrase “cell lineage” refers to the ancestry of a particular cell type, including ancestral cells and all of the subsequent cell divisions which occurred to produce the cell type. The phrase “cell status” refers to a state or condition of a cell at a given time. Exemplary combinations of markers expressed by the pulmonary stem cells are described in the examples with reference to human and mice pulmonary stem cells.

The pulmonary stem cells form individual colonies in vitro and are able to differentiate into a mature phenotype. The term “colony” refers to an aggregation of cells growing together as the descendants of a single cell, wherein an individual colony refers to a colony which is distinct from other aggregations in a cell culture. The term “cell culture” refers to the in vitro (i.e., outside of body, such as in a test tube or vat) propagation of cells isolated from living organisms. The term “differentiate” refers to the process cells undergo as the cells mature into a distinct cell type, wherein differentiated cells have distinctive characteristics, perform specific functions and are less likely to divide. The term “phenotype” refers to the total characteristics displayed by an organism, including eukaryotic or prokaryotic cells, unicellular and multicellular organism, under a particular set of environmental factors, resulting from interaction between the genotype and the environment, wherein a mature phenotype is the phenotype displayed by the organism complete in natural growth or development. Exemplary mature phenotypes of cells from a lung tissue are the phenotypes of alveolar type 2 and type 1 pneumocytes.

In mice, the pulmonary stem cells can be identified in vivo as scattered cells located at bronchoalveolar junctions of lung tissues. In in vitro cultures, the murine pulmonary stem cells form individual colonies, and express stem cell specific antigens including Oct-4 as well as markers of epithelial and Clara cell lineages and peroxiredoxin II (natural killer enhancing factor B).

In particular, isolated stem cells from mouse lungs in primary cultures express stem/progenitor markers Oct-4, SSEA-I, and Sca-1 as established by experimental procedures exemplified in Example 3. The murine pulmonary stem cells also express cytokeratin 7, a marker of epithelial cells, not detected in the surrounding cells of lung epithelium (see Example 3). Additionally, the murine pulmonary stem cells express Clara cell secretion protein and display cytochrome p450 activities (see Example 3). The murine pulmonary stem cells also express peroxiredoxin II and VI (see Example 3).

The murine pulmonary stem cells do not express other lung epithelial markers such as cytokeratin 5/8, 18 and 19 nor surfactant protein C and aquaporin-5, markers for alveolar type 2 and type 1 pneumocytes respectively (15, 16, 17, 18) (see Example 3). The murine pulmonary stem cells have the capacity to undergo terminal differentiation to alveolar type 2 and type 1 pneumocytes as established by experimental procedures exemplified in Example 4.

In in vitro primary culture, pulmonary stem cells from humans express stem cells specific antigens such as Oct-4⁺, SSEA-3⁺, SSEA-4⁺ and Sca-1⁺, as detected following procedures analogous to the ones exemplified in Example 3. The human pulmonary stem cells are also able to form colonies and also differentiate into mature phenotypes, as established by procedures analogous to the ones exemplified in Example 3. Any adjustment and/or modification in the experimental procedures herein described particular in Example 3 required by the use of human cells instead of murine cells and/or detection of markers other than the one mentioned in the examples is identifiable by a person skilled in the art upon reading of the present disclosure, in particular the Example section, and will not be described in further detail.

The identified pulmonary stem cells are target of various infective agents. The phrase “infective agent” refers to any material able of self-reproduction, also able to interfere with the normal functioning and/or survival of a cell. Infective agents include bacteria, parasites, fungi, viruses, prions, and viroids. As used herein, the term “interfere with” refers to specifically controlling, influencing or otherwise affecting the item indicated thereafter, and can include regulation by activation, stimulation, inhibition, alteration or modification; for example interfere with the normal functioning and/or survival of a cell refers to specifically controlling, influencing or otherwise affecting the normal functioning and/or survival of the cell and can include regulation by activation, stimulation, inhibition, alteration or modification of the normal functioning and/or survival of the cell.

In particular, the pulmonary stem cells are infected by viruses such as SARS-CoV, influenza, e.g. influenza A, as established by experimental procedures exemplified in Examples 5 and 6. The pulmonary stem cells are also infected with various types of avian flu such as HI1N2 and H3N2 as verifiable by procedures analogous to the ones exemplified in Examples 5 and 6. The pulmonary stem cells are selectively targeted by SARS-CoV as shown in Example 5, wherein SARS-CoV are shown to infect the pulmonary stem cells and not the cells surrounding the pulmonary colonies.

In some embodiments, the pulmonary stem cells can be used for the production of infective agents, e.g. viral particles, such as SARS-CoV as well as influenza and avian flu viral particles. In particular, in one embodiment exposure of the pulmonary stem cells to SARS-CoV leads to selective productive infection of the putative stem cells and the replication and release of infectious SARS-CoV particles as exemplified in Examples 5 and 6.

Accordingly, the method can comprise contacting an infective agent, such as a SARS virus particle with an isolated pulmonary stem cell in a culture; and collecting the SARS virus particles from the culture.

The term “contact” or “contacting” refers to placing the pulmonary stem cell and an infective agent or other biological agent, in a mutual spatial relationship such that a biological interaction between the infective agent, or biological agent and the pulmonary stem cell is feasible; the phrase “biological agent” refers to any material able to interfere with the biological condition of the pulmonary stem cells, which include protein, metabolites other compounds and biological material such as an infective agent; the phrase “biological interaction” refers to the process by which the biological agent interferes with the normal functioning and/or survival of the pulmonary stem cells. Contacting a SARS virus particle with an isolated pulmonary cell in a culture can be performed, by incubating a confluent primary culture with virus particles for a predetermined time, as exemplified in Examples 5 and 6, or by other methods identifiable by a person skilled in the art upon reading of the present disclosure.

The term “collect” or “collecting” refers to pick up or accumulate as if by harvesting. Collecting infective agents such as SARS virus particles from the culture can be performed by isolating supernatants of the cultures at predetermined times after infection as exemplified in Examples 5 and 6, or by other methods identifiable by a person skilled in the art upon reading of the present disclosure. The infective agent, such as SARS virus, can be identified in the supernatants by methods available in the art such as titration and/or immunofluorescence. Titration can be performed by plaque assay carried out on cells, such as Vero cells immunofluorescence can be performed using antibodies specific for the viral particles conjugated with a fluorescence agent. (see Examples 5 and 6). Analogous procedures can be performed for the production of other viruses such as influenza and avian flu viruses.

The amount of viral particles incubated, the time of incubation and of collection of the viral particles produced are predetermined based on the type of viral particles to be produced, the culture medium utilized, culture condition, desired amount of viral particles to be produced and/or other factors affecting the production of the viral particles in vitro, identifiable by a person skilled in the art. Exemplary procedures are illustrated in the Examples, in particular Examples 5 and 6.

Additional methods to perform the above mentioned steps can be envisioned by a person skilled in the art upon reading of the present disclosure, in particular the Examples section, and will not be further discussed in detail. Additional embodiments can be designed to produce infective agents other than SARS virus, in particular viruses such as influenza and avian flu viruses, wherein any adjustment and/or modifications of the procedures herein described required by the production of infective agents other than SARS virus, are identifiable by a person skilled in the art upon reading of the present disclosure, in particular the Examples section, and will not be further discussed in details.

In some embodiments, the pulmonary stem cells can be used to identify compounds able to interfere with infection of the pulmonary stem cells by an infective agent, for example SARS-CoV. To this purpose, one or more candidate compounds can be administered to the pulmonary stem cells in combination with the infective agent, e.g. SARS-CoV particles and the effect of the administration of the candidate compound on the ability of the infective agent to infect the cells, determined.

The ability of the infective agent to infect an isolated pulmonary stem cell can be determined by detecting, upon contact of the infective agent with the pulmonary stem cells, the expression of a marker associated with the infection of a cell by the infective agent. In embodiments, wherein the infective agent is SARS, exemplary markers associated with SARS virus infection are presence of vacuoles filled with viral particles in the cells, production of viral particles, and expression of a viral protein or nucleic acid associated with reproduction of the virus in the pulmonary stem cells. Other markers are identifiable by a person skilled in the art upon reading of the present disclosure.

As used herein, the term “interfere with an infection”, refers to specifically controlling, influencing or otherwise affecting an infection, and can include regulation by activation, stimulation, inhibition, alteration or modification of the process that leads to the infection. The term “infection” refers to the growth of an infective agent within the cell.

Accordingly, in the exemplary embodiments wherein the infective agent is SARS virus, the method can comprise contacting a candidate compound with isolated pulmonary stem cell; contacting the isolated pulmonary stem cell with SARS virus; detecting a level of SARS virus infection of the isolated pulmonary stem cell, and comparing the detected level of infection with a predetermined threshold value, the threshold value indicative of development of SARS infection of the cell.

Contacting a candidate compound with the isolated pulmonary stem cell can be performed by administering the compound in a manner and according to procedures known in the art to enable a biological interaction between the compound and the isolated pulmonary stem cell. A candidate compound as referred to herein can be a natural compound or a synthetically derived compound and include nucleic acid, proteins, a carbohydrates, lipids, and derivative thereof. Additionally, the candidate compound can have biological or chemical properties, such as totally or partially defined signal transduction regulatory properties. A candidate compound can be obtained by methods identifiable by a person skilled in the art. For example, a candidate compound can be derived by rational drug design or can be selected from libraries of natural synthetic or natural compounds, including chemical biochemical or combinatorial libraries. Exemplary candidate compounds are anti-idiotypic antibodies and/or catalytic antibodies, or fragments thereof.

Suitable procedures to contact a cell with a candidate compound in an effective manner can be accomplished by those skilled in the art based on variables such as, the conditions under which the compound is being administered, the type of cell being contacted and the chemical composition of the candidate compound (i.e., size, charge etc.) being administered. Contacting the isolated pulmonary stem cells with SARS virus can be performed by incubating SARS viral particles according to methods herein described and exemplified, and/or by additional procedures identifiable by a person skilled in the art upon reading of the present disclosure.

Detecting the levels of SARS infection of the isolated pulmonary stem cells can be performed by qualitatively and/or quantitatively determining the expression of one or more markers associated with SARS infection of the pulmonary stem cell, wherein such a quantitative and or qualitative determination results in a value, the value being a detectable property or aggregate of properties; exemplary values are the concentration of viral particles in supernatants of a cell culture or numbers of vacuoles including viral particles which are detectable in a cell.

The threshold value of SARS infection can be predetermined measuring the expression of a marker associated with development of viral infection in a significant population of cells and assessing, e.g. by a statistically significant experimental procedures, the value ranges associated to the occurrence of infection, and to non-occurence occurrence of infection. In light of this assessment, by comparing the two value ranges, it will then be possible to set a threshold value such that the above the threshold are indicative of the development of infection, and the values below such a threshold are indicative of non-occurrence of infection.

The above steps can be repeated in a statistically significant number of procedures to identify the effective amount of the compound, if any. The phrase “effective amount” of a compound refers to at least the minimum amount of a compound that is necessary to minimally achieve, and more preferably, optimally achieve, the desired effect (i.e. interference with infection of pulmonary stem cells by an infective agent). An effective amount for use in a given method can be readily determined by one skilled in the art without undue experimentation, depending upon the particular circumstances encountered (e.g. concentrations, type of infective agent and number, etc.).

Additional methods to perform the above mentioned steps can be envisioned by a person skilled in the art upon reading of the present disclosure and will not be further discussed in detail. Embodiments wherein the infective agent is an infective agent other than SARS, in particular viruses, such as influenza and avian flu viruses, can be performed by procedures analogous to the procedures herein described to SARS wherein any adjustment and/or modifications required by testing infective agents other than SARS virus, are identifiable by a person skilled in the art upon reading of the present disclosure, in particular the Examples section, and will not be further described in details.

According to an additional aspect, the isolated pulmonary stem cells can be used to identify an infective agent able to infect pulmonary cells. The method can comprise contacting a candidate infective agent with an isolated pulmonary stem cell; detecting the level of infection of the isolated pulmonary stem cell by the infective agent; and comparing the detected level of infection with a predetermined threshold level, the threshold level indicative of development of infection of the cell.

The level of infection and the threshold level can be determined by quantitative or qualitative analysis of the expression of one or more markers associated with infection of a cell by the candidate infective agent. Exemplary procedures for performing quantitative or qualitative analysis of the expression of markers associated with infection of a cell are herein described with reference to SARS and influenza viruses. Additional markers and methods to perform such a quantitative or qualitative analysis can be identified by a person skilled in the art based the nature and biology of the infective agent upon reading of the present disclosure and in particular of the Examples section.

Suitable procedure to contact a cell with an infective agent are exemplified in the present disclosure and in particular in the Examples section with reference to SARS and influenza viruses. Additional procedures to contact a cell with an infective agent on can be accomplished by those skilled in the art based on variables such as, type and biology of infective agent, conditions under which the infective is being administered, and the type of cell being contacted, upon reading of the present disclosure and in particular of the Examples section.

In some embodiments, the isolated pulmonary stem cells can also be used in treatment of various conditions associated with ARDS. In particular, the isolated pulmonary stem cells can be used to identify method of treatment of ARDS involving regeneration of damaged pulmonary tissue. In one embodiment, the isolated pulmonary stem cells can be delivered into lung tissues, by means of a bronchoscope or other means of aspirations to position cells to damaged tissues of individuals diagnosed with conditions associated with ARDS. Additionally or in the alternative, pulmonary stem cells can grow on biodegradable scaffold or polymers prior to delivery. Stem cells grown as artificial lung tissues can be used to test for efficacy of therapeutic agents intended for treatment of these individuals. These artificial tissues can also be used to identify specific pathogen strains, which these individuals are suffering from. Method to perform delivery of isolated pulmonary stem cells into lung tissues or grow isolated pulmonary stem cells on scaffold are identifiable by a person skilled in the art upon reading of the present disclosure and will not be further discussed in details.

According to a further aspect a method to identify adult stem cells in a tissue typically, comprising stem cell and non-stem cell, is disclosed. The term “identify” refers to discovering or determining the existence, presence, or fact of or otherwise detect an indicated item. The phrase “non-stem cells” refers to cells, of any type and in any state, which do not have the characteristic of stem cells. The adult stem cells can be in particular identified by the method for identifying stem cells in a tissue herein disclosed and exemplified for pulmonary stem cells in Example 1.

The method comprises identifying the cells of the tissue which are slowly dividing and express the stem cell marker Oct-4, wherein the slowly dividing Oct-4 expressing cells are able to form colonies in vitro, the identified slowly dividing Oct-4 expressing cells, upon isolation in primary culture, able to differentiate in a mature phenotype.

Identifying the cells of the tissue which are slowly dividing, can be performed by detecting a marker associated with slowly dividing cells. For example, identifying the slowly dividing cells of the tissue can be performed by detecting the cells that retain agents such as BrdU (see or example procedures extensively described in Example 1 for pulmonary tissue) for a time period that is based on the cellular turnover rate of the tissue investigated. For murine pulmonary tissues disappearance of BrdU can be monitored for a time period of from about 1 week to about 5 weeks, in particular 4 weeks (see Example 1). Other tissues may require a different time of observations which could be days or weeks.

Identifying Oct-4 expressing cells can be performed by detecting the expression of marker Oct-4 in the cells using any suitable identifier and methods recognizable by a person skilled in the art upon reading of the present disclosure. Exemplary identifiers, such as antibodies and oligonucleotides, and exemplary methods such as immunofluorescence, RT-PCR and quantitative real time PCR, are shown in the examples (see Examples 1 and 3).

In some embodiments, identifying the cells which are slowly dividing and express marker Oct-4 can be performed by identifying slowly dividing cells and identifying the slowly dividing cells that express Oct-4 marker, as exemplified in Examples 1 and 3.

An identifier of slowly dividing cells and an identifier of the above listed markers can be included in a kit of parts for the identification of stem cells in a tissue.

In one embodiment the kit of parts can comprise a first identifier for the identification of slowly dividing cells and a second identifier for the identification of the expression of Oct-4 marker, the first and the second identifier to be used in any the methods to identify stem cells herein disclosed. In particular, the first and the second identifier to be used in a method for identifying stem cells of a tissue, wherein the first identifier is used to identify slowly dividing cells of the tissue and the second identifier is used to identify the cells expressing Oct-4 marker of the tissue, wherein the identified slowly dividing cells expressing Oct-4 marker, which are able to form colony in vitro and to differentiate into an mature phenotype are the stem cells of the tissue.

The kit can further include a third identifier for identification of tissue stem cells specific markers, such as SSEA-1 and Sca-1 for murine pulmonary stem cells and SSEA-3, SEEA-4 and Sca-1 in human pulmonary stem cells. The kit can further include a fourth identifier of the mature cell phenotype.

The first, second, third and fourth identifiers can be provided in the kits, with suitable instructions and other necessary reagents, in order to perform the methods herein disclosed. The kit will normally contain the identifier in composition included in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

Further details concerning the identification of the identifier additional component to included in the compositions, and generally manufacturing and packaging of the kit, can be identified by the person skilled in the art upon reading of the present disclosure.

Additional kits of parts for performing at least one of the other methods herein disclosed, the kit comprising suitable identifiers, compositions and reagents for performing such methods can be identified by a persons skilled in the art and will not be further described in details.

The identified slowly dividing Oct-4 expressing cells of the adult tissue can be then tested for the ability of forming colonies and differentiate into a mature phenotype, by procedures herein described and/or identifiable by a person skilled in the art upon reading of the present disclosure. To this extent, the tissue cells can be cultivated in vitro according to any suitable procedure to cultivate the tissue cell identifiable by a person skilled in the art upon reading of the present disclosure, based on the type of cells to be cultivated. The term “cultivate” refers to a procedure performed to grow cells in vitro.

Additional embodiments of the method to identify adult stem cells wherein the above steps are performed in a different order and/or by additional procedures can be identified by a person skilled in the art and will not be further described in details.

In some embodiments, the adult stem cells can be cultivated according to a method for cultivating adult stem cells in vitro herein disclosed. The method can comprise: providing a tissue comprising tissue cells, isolating the tissue cells from the tissue. The term “isolate” refers to a procedure to separate the tissue cells from other tissue material. The tissue cells can be isolated by treating the tissue with appropriate procedure to release the tissue cells such as the ones extensively described in Example 2.

The released tissue cells can be then incubated in a suitable medium for a variable time which can be determined on the basis of the type of cells to be cultivated and the desired number of cell colonies to be formed. The term “incubate” refers to place the cells under favorable conditions to grow and develop.

The term “medium” refers to a substance used to provide nutrients for cell growth, according to a technology to artificially cultivate cells, of mammal origin, in a laboratory or production-scale device (i.e., in vitro). It may be liquid (e.g., broth) or solid (e.g., agar) and may include special growth media (fluids that bathe the cultured cells with the right amounts of amino acids, salts, and other minerals). For pulmonary stem cells the time period can be from 5 to 15 days.

The medium can be selected on the basis of the nature of the tissue and cells to be cultivated, and the incubation performed according to procedures identifiable by a person skilled in the art and preferably including procedures suitable to enrich the tissue cells, such as centrifugation and resuspension in appropriate medium (see Example 2).

The method further comprise applying a cell sorting procedure such as a flow cytometric sorting procedure to the incubated tissue cells to obtain colonies in primary culture according to procedures herein extensively described in Example 2 for pulmonary tissue, wherein any adjustment or modification for tissue cells other than pulmonary tissue cells is identifiable by a person skilled in the art upon reading of the present disclosure. The term “sorting procedure” refers to a process utilized by a user (e.g., by researchers) to sort/separate different cells; automated means of cell sorting include “biochips” (utilizing controlled electrical fields to collect specific cell types onto electrodes in the biochip), fluorescence-activated cell sorter (FACs) machines, magnetic particles (e.g., attached to antibodies), etc.

The colonies so obtained can then be screened to identify the colonies including Oct-4 expressing cells able to differentiate into a mature type, as herein described.

Additional embodiments of the method to cultivate adult stem cells in vitro can be envisioned by a person skilled in the art upon reading of the present disclosure and in particular the Examples section and will not be further described in details.

The following examples are provided to describe the invention in further detail. These examples, which set forth a preferred mode presently contemplated for carrying out the invention, are intended to illustrate and not to limit the invention.

### EXAMPLE 1

### Localization of BRDU-Retaining OCT-4 Expressing Cells in Mice Lungs

a. Localization of BrdU Retaining Cells in Mice Lungs

Neonatal ICR mice were injected intraperitoneally with 50 mg/kg of 5-bromo-2′-deoxyuridine (BrdU) (Sigma) at 12.5 mg/ml in phosphate-buffered saline (PBS) twice a day for five days. Mice were then maintained without additional BrdU injection and sacrificed on day 0, weeks 1, 2, 3 and 4 after injection. Lungs were removed, fixed in 10% formalin fixative and embedded in paraffin. The paraffin-embedded sections were then subjected to immunohistochemical analysis.

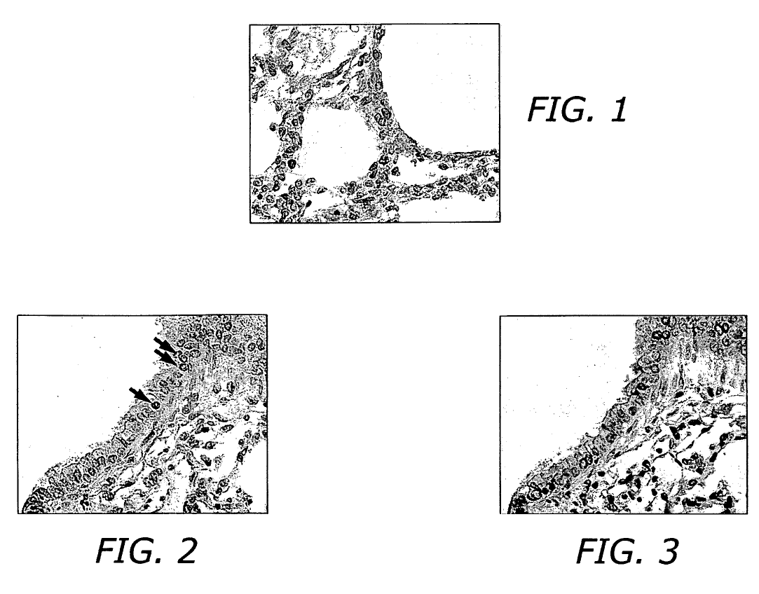

The paraffin-embedded sections were dewaxed and re-hydrated. BrdU staining was performed as described (21) with anti-BrdU-monoclonal antibodies (M 0744, Dakocytomation) at 1:100 and a peroxidase detection kit (Vector VIP Substrate Kit, Vector) with diaminobenzene (DAB) as substrate according to manufacturer's instructions. Results are shown in FIGS. 1 to 3.

At day 0, actively dividing, cells including some alveolar cells, in the lung demonstrated nuclear staining for BrdU, (FIG. 1). At week 2 and week 3 after labeling, the number of BrdU positive cells significantly declined (data not shown). At week 4 after labeling, only a few cells among the epithelial cells still retained BrdU labeling (see FIG. 2 wherein the BrdU retaining cells are also indicated by arrows). The localization of the BrdU retaining cells in the bronchoalveolar area was confirmed by staining performed with monoclonal antibodies against pan-cytokeratin (FIG. 3) (14).

b. Localization of Oct-4 Expressing Eells in Mice Lungs

Paraffin-embedded section of lung removed 4 weeks after labeling with BrdU were also tested for detection of Oct-4-expression, as described in (22), wherein antigen retrieval for tissue sections was carried out by heating in 10 mM sodium citrate buffer (pH 6.0) for 8 min, with the tissue sections then incubated for another 15 min in room temperature. The tissue sections were incubated overnight at 4° C. with the primary antibodies directed against Oct-4 (sc-908I, Santa Cruz Biotechnology), followed by peroxidase detection. Sections were counterstained with Mayer's hematoxylin to mark unstained nuclei.

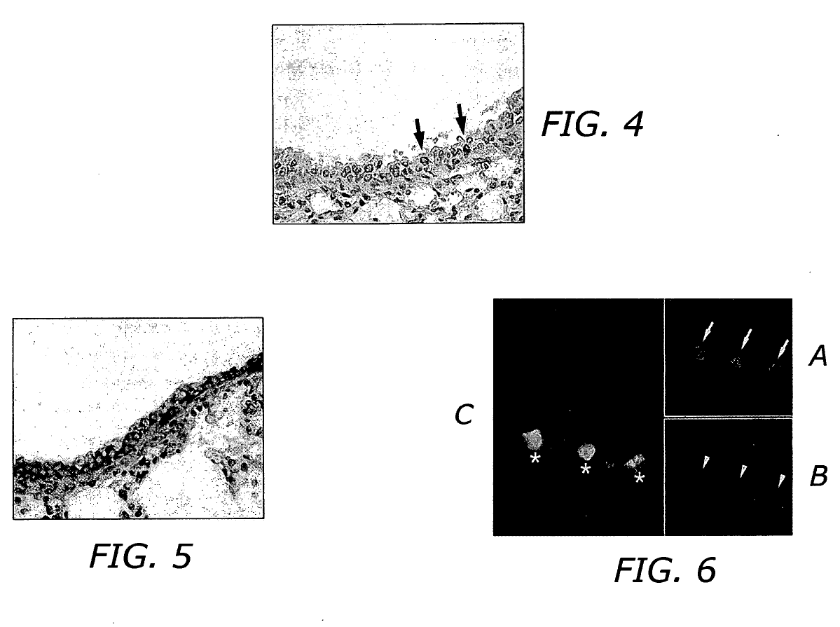

The results of this series of experiments are shown in FIGS. 4 and 5. A small number of cells located in the same region where BrdU retaining cells were identified (see FIG. 3) were also found to be positive for Oct-4 (FIG. 4) wherein localization of the Oct-4 positive cells in the bronchoalveolar area was confirmed by immunostaining performed on the same section with anti-pancytokeratin antibodies (FIG. 5).

c. BrdU Retaining Cells Also Axpress Oct-4

Sections of mice lungs taken 4 weeks after labeling with BrdU were tested for double staining with anti-BrdU and Oct-4 antibodies. Tissue sections were treated as described in sections a. and b. above, wherein the following fluorescence-labeled secondary antibodies were used: Cy3-labeled F(ab′)₂ goat anti-rabbit IgG (H+L) and FITC-labeled F(ab′)² goat anti-mouse IgG (H+L) (Jackson Immuno Research).

The results shown in FIG. 6, confirm the presence of BrdU retaining cells (see red-stained cells of FIG. 6A) and Oct-4 expressing cells (see green-stained cells of FIG. 6B) in mice lungs 4 weeks after labeling. Additionally, this set of experiments also demonstrates co-localization of Oct-4 expression with BrdU retention in mice lungs 4 weeks after labeling (see merged images of BrdU retaining cells of FIG. 6A and Oct-4 expressing cells of FIG. 6B shown in FIG. 6C wherein the merged images of BrdU retaining cells and Oct-4 expressing cells are indicated by asterisks).

These results show the presence of slow cycling, Oct-4 expressing epithelial cells in the terminal bronchioles adjuvant to alveolar sacs in lung tissues.

### EXAMPLE 2

### Primary Cell Cultures of BRDU-Retaining/OCT-4 Expressing Clonogenic Lung Cells

To better characterize the rare BrdU retaining Oct-4 expressing cells identified following the procedure exemplified in Example 1 above, the inventors developed a procedure to cultivate those lung cells in vitro.

a. Process to Derive Primary Cells Cultures of BrdU-Retaining/Oct-4 Expressing Clonogenic Lung Cells

Neonatal ICR mice were sacrificed by cervical dislocation. Lungs were removed, cut into small pieces and suspended in Hank's buffer containing penicillin and streptomycin. The lungs were washed five times and then incubated with 0.1% protease type XIV (Sigma) in minimum essential medium Eagle-Joklik modification (JMEM) (Sigma) at 4° C. for 12 h. Afterward, tissues were transferred to JMEM containing 10% FCS and pipetted several times to release pulmonary cells.

After filtering through a 100-μm nylon cell strainer (BD Biosciences), the released cells were washed and re-suspended in MCDB-201 medium supplemented with insulin (5 μg/mL), transferrin (5 μg/mL), and epidermal growth factor (10 ng/mL). The lung tissue from one neonatal mouse could yield approximately 1.0-1.5×10⁶ cells in this digestion procedure. Cells were cultivated at a concentration of 4.5×10⁵ /mL per well in 12-well Petri dishes which were coated with type I collagen (10 μg/cm²).

Pulmonary cells were isolated from lung tissue from neonatal mice and grown in MCDB-201 medium supplemented with insulin, transferrin, and epidermal growth factor.

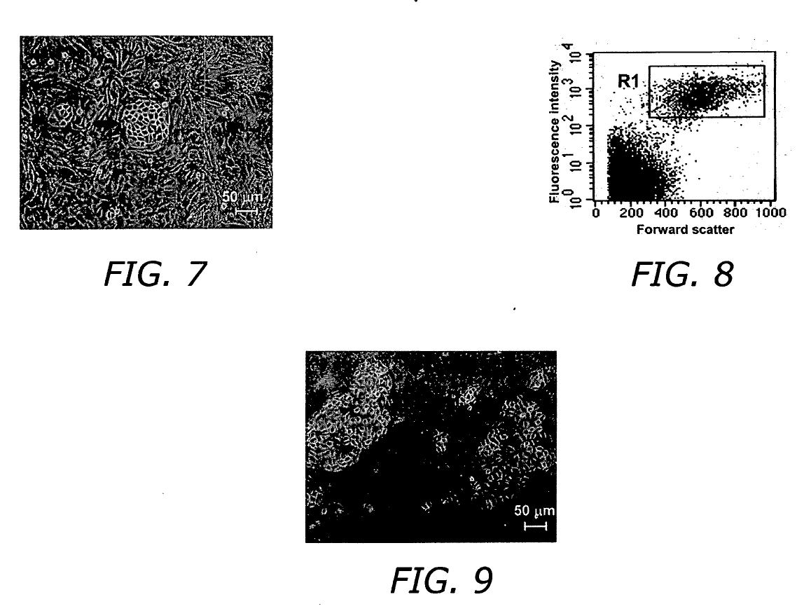

After 5-7 days of culture, a phase-contrast photograph for primary culture of lung tissue from neonatal mouse was taken as shown in FIG. 7. The phase contrast photograph shows small, morphologically recognizable colonies of approximately 50 cells (see arrows in FIG. 7).

Cells in the colonies shown in FIG. 7, were densely packed, highly reflectile, and easily distinguishable from the surrounding spindle shaped cells under phase contrast microscopy. Since the incidence of such colonies was very low, a flow cytometric sorting procedure was developed to enrich the number of these colony forming cells.

b. Flow Cytometric Sorting of Pulmonary Cells

Cells isolated from lung tissue as reported in section a. of this example, were first incubated with 2-bromoacetamidoethyl sulfonamide, (Dapoxyl®), a flurogenic supravital dye for the measurement of cellular thio content, then excited at 374 nm and afterwards sorted for both high forward scatter and fluorescence at 572 nm.

Results shown in FIG. 8 illustrate the presence of a subpopulation of cells with high fluorescence intensity, identified in the diagram as R1 region defined for both fluorescent intensity and forward scatter. Accordingly, primary culture of the sorted cells, which is comprised 8 to 10 % of total cell suspension, yielded numerous colonies ranging in size from a few cells to a few hundred cells after ten days of culture. Using ≧50 cells as criteria for a colony, there were 106±5 colonies per 10⁵ sorted cells plated. The frequency of these clonogenic cells was estimated to be approximately 0.008-0.011% of all nucleated cells isolated from lung tissues.

c. Enrichment of Pulmonary Colony Forming Cells

Pulmonary cells obtained from lung tissues as reported in section a. and b. of this example, were incubated at 1×10⁷ cells/mL in MCDB-201 medium with 2-bromoacetamidoethyl sulfonamide (Dapoxyl) at a final concentration of 2.5 μM. After incubation for 5 mm at 37° C., cells were centrifuged and re-suspended in MCDB-201 with 5% FCS (v/v).

Fluorescence-activated cell sorting was carried out with a FACSvantage SF machine (BD Biosciences), using an Enterprise II laser to generate UV lines for excitation. The fluorescence was collected using a 505-nm long-pass (LP) filter. Target cells were sorted into 1 mL of MCDB-201 medium supplemented with insulin, transferrin and epidermal growth factor. Cells were cultivated at 1-1.5×10⁵/mL and many colonies appeared after 10 to 14 days in culture. These colonies were comprised of at least 50 cells to hundreds of cells in a single colony.

The results illustrated in FIG. 9 show many large pulmonary epithelial colonies appearing after enrichment (see arrows in FIG. 9). In particular, primary culture of sorted cells, comprising 8 to 10% of total cells, yielded numerous colonies ranging in size from few cells to a few hundreds cells after ten days of culture (FIG. 9).

One neonatal mouse yields approximately 1.0-1.5×10⁶ cells in our procedure. In our sorting experiments, approximately 8 to 10% of the total cells (1×10⁷) used were collected and the cells were cultivated at 1×10⁵ cells/plate on collagen-1 coated plate (a total of about 10 plates). For data reported, three separate experiments were performed. For each experiment, 8 to 10 wells were estimated. And using ≧50 cells as criteria to count for a colony, the number of colonies were analyzed statistically. The number of the colonies per 1×10⁵ sorted cells was estimated to be 106±5. Accordingly, the frequency of these clonogenic cells was estimated to be approximately 0.008-0.011% of all nucleated cells isolated from lung tissues.

### EXAMPLE 3

### Primary Cell Cultures of BRDU-Retaining/OCT-4 Expressing Clonogenic Lung Cells Also Express SSEA-I and SCA-1

Pulmonary clonogenic colonies in primary cultures obtained as reported in Example 2, were examined for stem/progenitor markers by immunofluorescence using the embryonic and stem cell antigens, Oct-4, SSEA-I, and Sca-1, and by RT-PCR using probes for Oct-4 and Sca-1 mRNA.

a. Immunofluorescence

The pulmonary clonogenic cells were fixed in methanol:acetone (1:1) for 3 min at room temperature for Oct-4, and in 4% paraformaldehyde in PBS for 10 min at room temperature for other antigen determination. Afterwards, cells were then permeabilized in 0.1% Triton X-100 in blocking solution (3% BSA in PBS), washed three times, and left in blocking solution for 1 h. Cells were incubated at 4° C. overnight with primary antibodies. Primary antibodies used were as followed: aquaporin-5 (AB3069); c-Kit (CBL1359); cytokeratin 5/8, 7, and 18 (MAB3228, 3226, and 3234, respectively); pan-cytokeratin (AE1/3); pro-surfactant C protein (AB3786); SSEA-1 (MAB4301) (all from Chemicon International); cytokeratin 19 (IF15, Oncogene Research Products); AEC-2 (MAB933); Sca-I (AF1226), (both from R & D System); CCSP (sc-9773, Santa Cruz Biotechnology) and Oct-4.

The following fluorescence-labeled secondary antibodies were used (Jackson Immuno Research): Cy3-labeled F(ab′)₂ donkey anti-goat IgG (H+L); F(ab′)² goat anti-rabbit IgG (H+L); F(ab′)₂ goat anti-mouse IgG (H+L); F(ab′)₂ goat anti-mouse IgM, ychain specific; F(ab′)₂ goat anti-rat IgG (H+L); and FITC-labeled F(ab′)₂ donkey anti-goat IgG (H+L); and F(ab′)₂ goat anti-mouse IgG (H+L). Cell nuclei were counterstained with 4,6-diamidino-2-phenylindole (DAPI). Primary cultures were also analyzed for enzymatic activity of alkaline phosphatase, according to standard protocols using an AP detection kit (SCR004, Chemicon International).

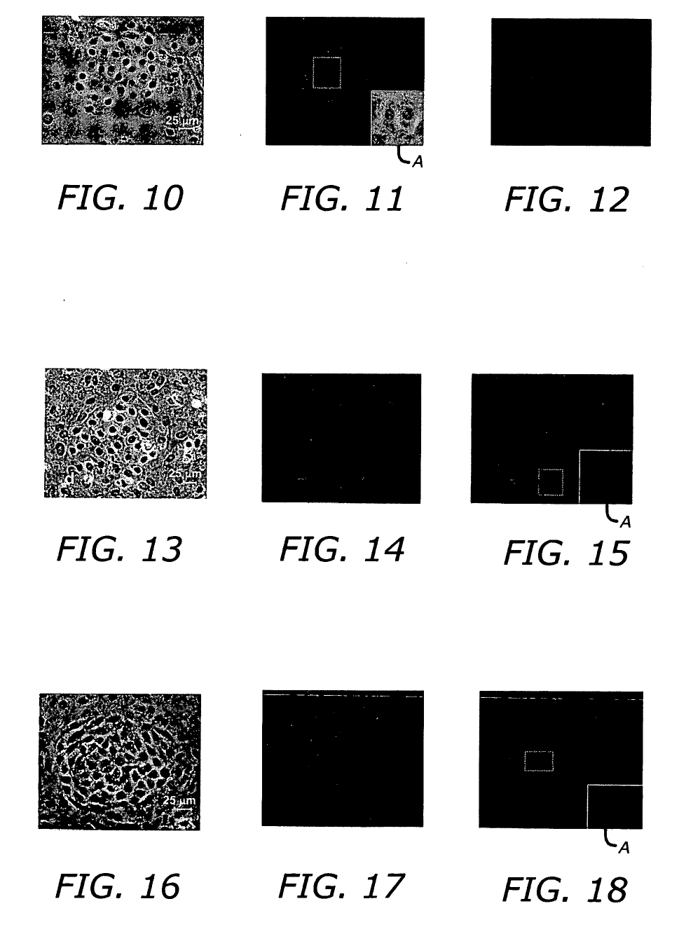

Immediately after culture, Oct 4 expression was too rare to be detected (picture not shown). However, following 5-10 days of culture, expression of Oct-4, SSEA-1 and Sca-1 were specifically detected in these pulmonary colony cells but not in the surrounding spindle shaped cells in the cultures as shown in FIG. 10-12 for Oct-4, FIG. 13-15 for SSEA and FIG. 16-18 for Sca-1.

In particular, Oct-4 expression was detected in the nuclei of these pulmonary colony cells (see panel A of FIG. 11 and FIG. 12) while SSEA-1 and Sca-1 were mainly found in the cell surface and cytoplasm (see FIGS. 14 and 17 and panel A of FIGS. 15 and 18).

Primary pulmonary cultures were also examined using specific antibodies directed against cytokeratin-7 and Clara cell secretion protein. The cells fixed with paraformaldehyde were permeabilized in 0.1% Triton X-100 containing 3% BSA in PBS. The primary antibodies for anti-cytokeratin-5/8, -7, -18, -19 and anti-Clara cell secretion protein antibodies were used to examine the pulmonary culture cells. Cells were counterstained with DAPI. Primary cultures were also analyzed for enzymatic activity of P450 as described in (16).

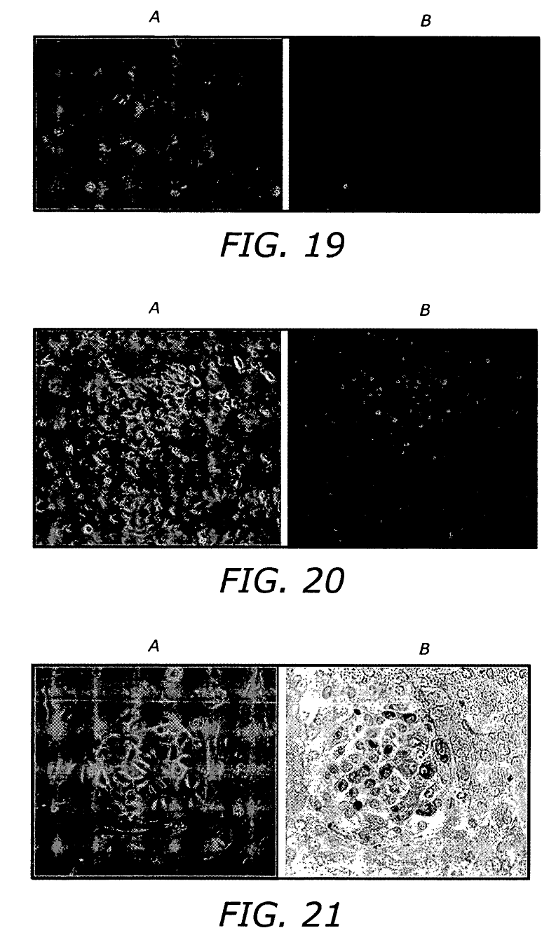

Results are shown in FIG. 19 (cytokeratin-7), FIG. 20 (Clara cell secretion protein) and FIG. 21 (cytochrome p450 enzyme), respectively. The results shown that only cytokeratin-7 were expressed only in the colony cells, however, other cytokeratin (e.g. cytokeratin-5/8, 18 and 19)could not be detected in the primary culture. The Clara cell secretion protein and cytochrome p450 enzyme activities were also detected in the colony cells.

They also expressed peroxiredoxin II and VI (data not shown), plasma levels of (which have been shown to be upregulated in response to infection with the SARS virus (19)).

Taken together, these findings suggest that these pulmonary colony cells exhibit phenotypes characteristics of stem/progenitor cells of the lung and also bear markers for epithelial and Clara cells.

b. RT-PCR

The presence of Oct-4 and Sca-1 mRNA in the primary culture of clonogenic lung cells was confirmed with RT-PCR of cells in the colonies individually plucked from culture described in section a. of this example. For Sca-1, the Vero E6 cells line was negative control and BW5147 cells line was positive control. For Oct-4, mouse testis Sertoli cells (TM4 cell line) were negative control; mouse embryonic germ cells were positive control.

Colonies were picked individually and pooled together. Under microscope, a 26 G needle was used to delimit the boundary of pulmonary colonies. Cells in the colonies were gently removed using a pipet tip from the dish. Twenty colonies with 2 to 3×10³ cells were collected for RT-PCR and approximately 1×10⁴ cells were used for Western-blotting. Total RNA was prepared with RNeasy Micro Kit (Qiagen) and cDNA generated with random primers.

For PCR reaction, the forward and reverse primers used were (i) the oligonucleotides having a sequence reported in the enclosed sequence listing as SEQ ID NO: 1 and SEQ ID NO: 2 for Oct-4 expression (23) (ii) the oligonucleotides having a sequence reported in the enclosed sequence listing as SEQ ID NO: 3 and SEQ ID NO: 4 for Sca-I expression (24); and (iii) the oligonucleotides having a sequence reported in the enclosed sequence listing as SEQ ID NO: 5 and SEQ ID NO: 6 for GADPH expression as the control. The PCR was carried out at (i) 94° C. for 1 mm, 54°C. for 1 min, and 72° C. for 3 min for 35 cycles (for Oct-4) and (ii) 94° C. for 1 min, 48° C. for 1 min, 72° C. for 1 min for 35 cycles (for Sca-1).

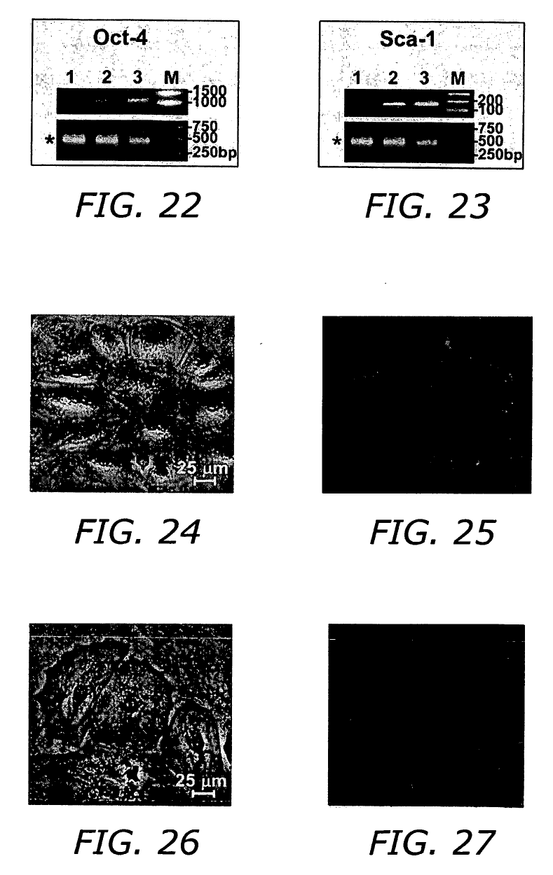

The results are shown in FIG. 22 and 23. The bands for Sca-1 and Oct-4 were consistent with the expected size of 160 bp and 1121 bp, respectively. The GADPH was used as internal standard for both reactions. The presence of Oct4 and Sca-1 mRNA was confirmed with RT-PCR in the cells plucked from individual colonies.

### EXAMPLE 4

### Primary Cell Cultures of BRDU-Retaining/OCT-4 Expressing are Able to Differentiate in Alveolar Pneumocytes

To investigate whether the pulmonary clonogenic colonies in primary cultures obtained as reported in Example 3 (pulmonary colony cells) could differentiate to form lung tissue subtypes, individual colonies were plucked from primary culture, transferred to new collagen I-coated dishes, and cultured in conditioned media harvested from 5-7 day pulmonary cell cultures.

At different times after colony transfer into dishes free from the surrounding environment of spindle shaped cells, the cells originated from individual pulmonary colonies were examined for morphology, and for Oct-4, and pneumocyte markers. In particular, the analysis was performed at day 1, 5, and 9 after colony transfer. Results are shown in FIGS. 24 to 25.

On day 1, Oct-4 expression was still detectable in most of the subculture cells, similar to the original pulmonary colonies (not shown).

By day 5, Oct-4 expression became undetectable in all but a few remaining round cells with Oct-4 expression (data not shown). These cells without Oct-4 expression gradually flattened and became oligonal, less reflective cells (FIG. 24), a morphological appearance similar to that of type 2 pneumocytes. They also began to express surfactant protein-C, a marker for type 2 pneumocytes (17), in the cytoplasm, especially in the perinuclear region (FIG. 25).

By day 9, the diameter of the cells continued to increase, to approximately five-fold their original diameter (FIG. 26). Expression of surfactant protein-C decreased, while expression of aquaporin-5 protein, a marker for type 1 pneumocytes (18), became pronounced (FIG. 27).

These results show that the cells in the pulmonary colonies have the capacity to undergo differentiation into phenotypes characteristics of the differentiated alveolar pneumocytes.

### EXAMPLE 5

### Primary Cell Cultures of BRDU-Retaining/OCT-4 Expressing Cells Are Target of SARS CoV Infection

In order to investigate whether primary pulmonary cell cultures might be susceptible to SARS-CoV infection, we expose these primary cultures to SARS-CoV infection. All experiments for SARS-CoV infection were carried out at a P4 facility.

a. Primary Cell Culture BrdU-Retaining/Oct-4 Expressing Cells Are Target of SARS Co-V Infection

5-7 days old confluent primary cultures of pulmonary cells with epithelial colonies were incubated with SCAR-CoV (strain Tw7) at MOI of 0.5 (25) for 8 h and 24 h at room temperature. Cells were washed once with PBS buffer and incubated in MCDB-201 medium containing supplements. At 8, 16, 24 and 48 h postinfection, the supernatant of cells was collected for titration. The cells were then washed three times with PBS buffer and fixed with ethanol:acetone (1:1) before immunofluorescence analysis. The SARS-CoV antigen was detected using a mouse monoclonal antibody (diluted 1:2000) generated against the recombinant SARS-CoV nucleocapsid protein for this study. The epitope of this monoclonal antibody was shown to be localized to the N-terminal of the nucleocapsid (data not shown).

The titers of virus were determined by serial dilutions and analyzed by the monolayer procedure of plaque assay, which was modified from Burleson (26). After Vero-E6 reached confluence on 6-well tissue culture plate, 0.5 ml of appropriate virus dilutions were prepared and applied onto cells. Followed the absorption for 30 min at 37° C., the un-adsorbed virus was removed and overlaid 1 % of agar with DMEM and 2% FBS. Until the plaques formed about four days, the agar overlay was removed and the cells were stained with crystal violet solution (10%) for 10 min. Finally the plaques could be counted and the titer can be calculated by dilution fold and the sample volume used. For bioactivity assay, a confluent VeroE6 cell culture was mixed with the SARS-CoV particles which collected from the supernatant of primary culture cells at 2 MOI for 24 h at room temperature. The procedures for virus identification were described above.

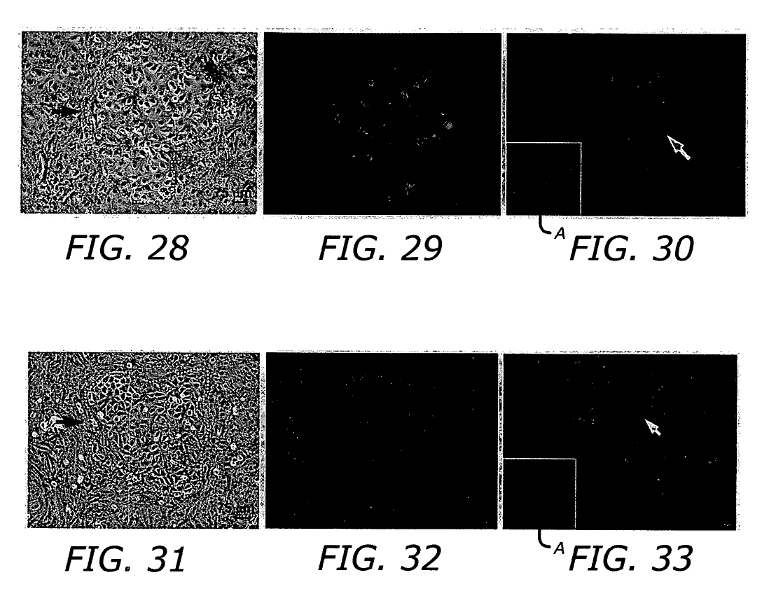

Results are shown in FIG. 28 to 30 (8 h incubation) and in FIGS. 31 to 33 (24 h incubation).

At eight hours of incubation, approximately 30% of cells within the pulmonary colonies displayed strong immunofluorescence for SARS-CoV (FIG. 28-c). The percentage of SARS-CoV positive pulmonary colony cells rose to approximately 60% at sixteen hours, and by 24 hours, nearly every cells in the pulmonary colonies was positive for SARS-CoV infection (FIG. 32). The pulmonary colony cells began to detach and exhibited cytopathic changes by 48 h (data not shown). In contrast, none of the cells surrounding the pulmonary colonies became infected at any time point examined (FIG. 30 and 33).

b. Primary Cell Culture BrdU-Retaining/Oct-4 Expressing Cells Are Not a Selective Target of Influenza Infection

Cultures containing pulmonary colony cells were infected with influenza A virus, at a MOI of 0.5 for 8 hours under the same conditions described in section a. of this example.

The influenza A/WSN/33 virus was propagated and maintained as described (27) (generous gift from Dr. Shieh-Shin-Ru at Chang-Gung University). Infections with the influenza A virus were performed similarly to SARS-CoV infection at same MOI. Cells were incubated for 8, 16 and 24 h postinfection and examined for evidence of infection as determined by immunofluorescence using a viral nucleoprotein specific antibody from an influenza detection kit (IMAGEN™ Influenza virus A and B, DakoCytomation).

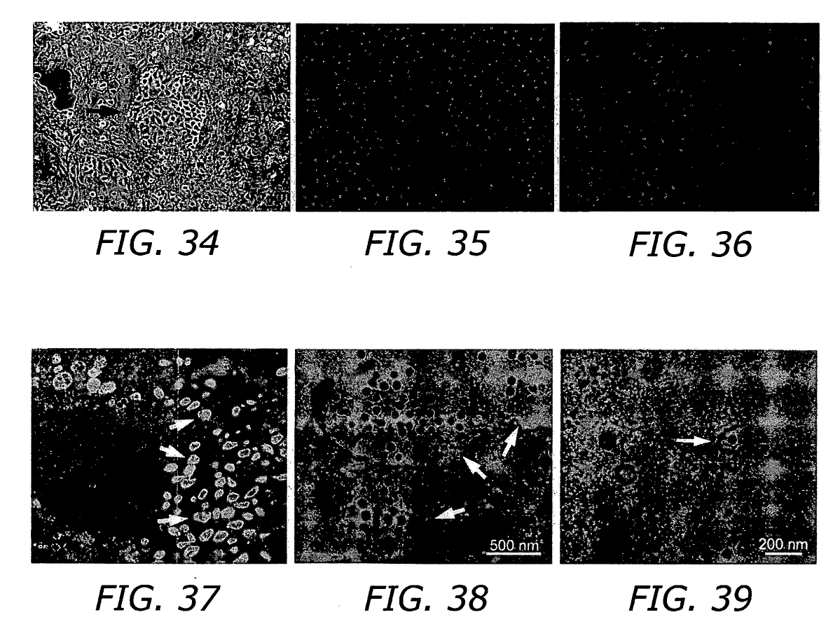

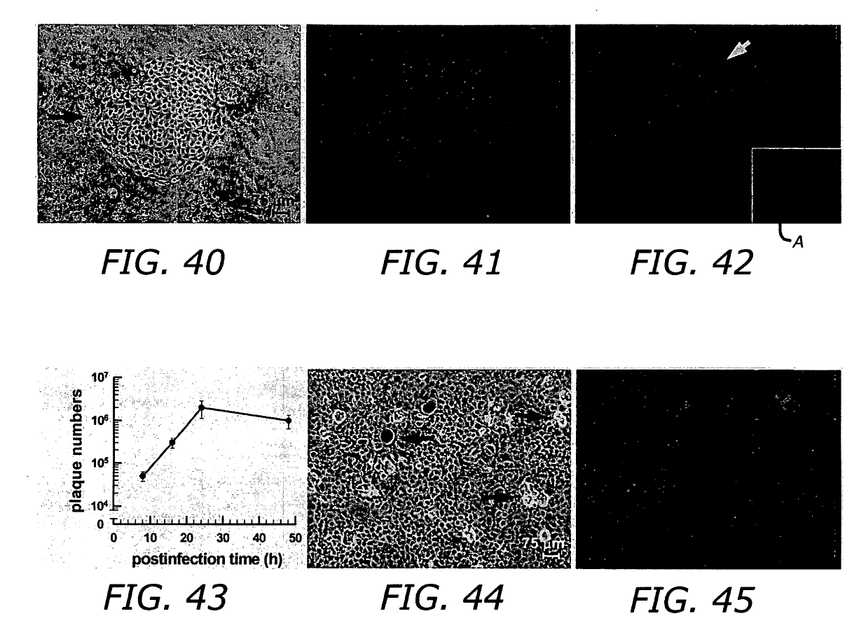

Results are shown in FIGS. 34 to 36. In contrast to SARS-CoV, influenza A virus infected all cells without preference for pulmonary colony cells (FIG. 34-36) and cytopathic effects were observed in the cultured cells as early as at 24 hours after infection (picture not shown). Overall, these experiments demonstrated that pulmonary stem cells are selectively susceptible to SARS-CoV infection.

### EXAMPLE 6

### Primary Cell Cultures of BRDU-Retaining/OCT-4 Cells Expressing Are Selected Target of SARS-CoV Infection

To investigate whether SARS-CoV specifically target undifferentiated pulmonary stem cells the following experiments were performed.

First, immediately after cell isolation from lung tissues, pulmonary cell suspensions were incubated with SARS-CoV at a range of MOI from 0.5 to 10 and assayed for their ability to become infected by immunofluorescence staining for SARS-CoV nucleocapsid protein. No nucleocapsid staining above background fluorescence was observed.

Next, cultures of pulmonary stem cells that had undergone differentiation in vitro into type 1 and type 2 pneumocytes (as discussed in example 4 above and shown in for FIG. 18 and n) were incubated with SARS-CoV at the same 0.5 to 10 MOI. These in vitro differentiated mature pneumocytes were found not to be susceptible to infection under the same conditions.

Afterwards, electron microscopy was performed to confirm the presence of actively replicating virus in the cytoplasm of the infected cells of the pulmonary colonies. For ultrastructural analysis, the lung cells were cultivated on an embedding/cell growing film (Aclar-fluoropolymer films, Structure Probe) which were coated with type I collagen. The culture conditions were described in the example 3 above. The pulmonary epithelial cells were infected with SARS-CoV at 0.5 MOI.

Sixteen-hours after infection, the fixation of pulmonary epithelial monolayer culture was first carried out in 2.0% glutaraldehyde and 4.0% paraformaldehyde in PBS for 2 h, post-fixed in 1% osmium tetroxide for 1 h. The cells were dehydrated in graded ethanol, washed in propylene oxide and infiltrated in a 1:1 mixture of propylene oxide and Spurr's resin (Sigma-Aldrich). Cells were then embedded in Spurr's resin and polymerized for 24 hours at 70° C. Sections were cut and stained with aqueous uranyl acetate and lead citrate. The ultrathin sections of infected cells were observed through transmission electron microscopy (Hitachi H-7000). Results are shown in FIG. 37 to 39.

At sixteen hours after the infection, the.cytoplasm of infected cells contained numerous swollen empty sacs around perinuclear region. (FIG. 37). At higher magnification, many enlarged swollen vacuoles filled with virus particles were observed (FIG. 38). On the outside of the cells, mature virus particles were observed and some of these extracellular virus particles were seen to associate with coated pits (FIG. 39) (20).

In order to ascertain whether SARS-CoV replicated and produced infectious virus particles in the cells of pulmonary colonies, culture media were collected at different time points after infection and assayed for infectious SARS-CoV using VeroE6 cells. Results are shown in FIG. 43 to 45.

As shown in FIG. 43, infected pulmonary yielded culture media with the increasing SARS-CoV infectivity: the titers of these culture media after 8, 16, 24 and 50h postinfection for VeroE6 infection were respectively, 5, 30, 210 and 104×10⁴ plaques). VeroE6 cells infected 16 to 48 hours with conditioned media demonstrated cytopathic alterations and SARS-CoV nucleocapsid protein immunostaining in all cells in the culture dish (FIG. 44-45). These experiments demonstrated that SARS-CoV maintained its replicative activities in the pulmonary colonies.

In summary, these experiments demonstrate the existence of a rare subpopulation of slow cycling pulmonary stem cells at the bronchoalveolar junction of the neonatal lung. They are capable of forming colonies in vitro, and continuously express stem cell markers such as Oct 4, SSEA-1 and Sca 1 antigen, and can differentiate to form type 1 and type 2 pneumocytes upon clone transfer. In addition, stem cells also express markers known to be expressed in epithelial and Clara cell lineages, which have been implicated in pulmonary repair and regeneration.

Exposure to SARS-CoV leads to selective productive infection of the stem cells and the replication and release of infectious SARS-CoV particles. In contrast, the alveolar pneumocytes either in the initial cell suspensions prepared from the lung tissues or those differentiated in vitro from the primary colony cells were resistant to SARS-CoV infection.

The finding that the above mentioned stem cells express peroxiredoxin II and VI may be of clinical significance. Peroxiredoxins were originally identified as intracellular proteins with multiple functions including enhancing natural killer cell activity, increasing resistance to oxidative stress, regulating transcription activator proteins, and providing antiviral activity against HIV. A recent proteomic analysis of plasma samples from patients with SARS demonstrated that plasma levels of peroxiredoxin II, are significantly elevated in patients with SARS (19).

These findings support the notion that pulmonary stem cells may be an important target for SARS-CoV infection. Loss of this pulmonary subpopulation may thus compromise the ability of lung tissue to recover from initial injury, and may help explain the late phase of clinical deterioration in SARS patients which is associated with significant morbidity and mortality.

In summary, isolated pulmonary stem cells, the isolated pulmonary stem cells being slowly dividing Oct-4 expressing cells forming individual colonies and able to undergoing terminal differentiation into a mature phenotype in vitro; a method to identify stem cells comprising: identifying slowly dividing Oct-4 expressing the cells of a tissue able to form colonies in vitro, and to differentiate in a mature phenotype; a kit for the identification of stem cell of a tissue, comprising a first identifier to identify slowly dividing cells and a second identifier to identify Oct-4 expression in the cells; a method for cultivating adult stem cells in vitro, comprising: incubating isolated tissue cells in a suitable medium; applying to the isolated incubated cell a cell sorting procedure to obtain colonies; and selecting the Oct-4 expressing colonies, the colonies able to differentiate into mature type; a method for identifying an infective agent able to infect pulmonary cells comprising: contacting a candidate infective agent with an isolated pulmonary stem cell; detecting the level of infection of the isolated pulmonary stem cell by the infective agent; and comparing the detected level of infection with a predetermined threshold level, the threshold level indicative of development of infection of the cell; a method for the production of a virus able to infect pulmonary cells, the method comprising: contacting a virus particle with an isolated pulmonary stem cell according in a pulmonary stem cell culture; and collecting virus particles from the culture. A method for identifying a compound interfering with infection of a cell by an infective agent, the method comprising: contacting a candidate compound with an isolated pulmonary stem cell; contacting the isolated pulmonary stem cell with a virus; detecting the level of the virus infection of the isolated pulmonary stem cell; and comparing the detected level of infection with a predetermined threshold level, the threshold level indicative of development of virus infection of the cell.

The disclosures of each and every publication and reference cited herein are incorporated herein by reference in their entirety.

While the cells, methods and kits have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

### REFERENCES

1. Ksiazek, T. G. et al. A novel coronavirus associated with severe acute respiratory syndrome. N. Engl. J. Med. 348, 1953-1966 (2003).

2. Drosten, C. et al. Identification of a novel coronavirus in patients with severe acute respiratory syndrome. N. Engl. J. Med. 348, 1967-1976 (2003).

3. Peiris, J. S. et al. Clinical progression and viral load in a community outbreak of coronavirus-associated SARS pneumonia: a prospective study. Lancet 361, 1767-1772 (2003).

4. Fowler, R. A. et al. Critically ill patients with severe acute respiratory syndrome. JAMA 290, 367-373 (2003).

5. Joynt, G. M. et al. Late-stage adult respiratory distress syndrome caused by severe acute respiratory syndrome: abnormal findings at thin-section CT. Radiology 230, 339-346 (2004).

6. Otto, W. R. Lung epithelial stem cells. J. Pathol. 197, 527-535 (2002).

7. Hong, K. U. et al. In vivo differentiation potential of tracheal basal cells: Evidence for multipotent and unipotent subpopulations. Am. J. Physiol. Lung Cell Mol. Physiol. 286, L643-L649. (2004).

8. Borthwick, D. W. et al. Evidence for stem-cell niches in the tracheal epithelium. Am. J. Respr. Cell Mol. Biol. 24, 662-670 (2001).

9. Van Winkle, L. S. Cellular response in naphthalene-induced Clara cell injury and bronchiolar epithelial repair in mice. Am. J. Physiol. 269, L800-L818 (1995).

10. Ten Have-Opbroek, A. A. et al. The alveolar type II cell is a pluripotential stem cell in the genesis of human adenocarcinomas and squamous cell carcinomas. Histol Histopathol. 12, 319-336 (1997).

11. Hong, K. U. et al. Clara cell secretory protein-expressing cells of the airway neuroepithelial body microenvironment include a label-retaining subset and are critical for epithelial renewal after progenitor cell depletion. Am. J. Respir. Cell Mol. Biol. 24, 671-681 (2001).

12. Reynolds, S. D. et al. Neuroepithelial bodies of pulmonary airways serve as a reservoir of progenitor cells capable of epithelial regeneration. Am. J. Pathol 156, 269-278 (2000).

13. Giangreco, A. et al., Terminal bronchioles harbor a unique airway stem cell population that localizes to the bronchoalveolar duct junction. Am. J. Pathol 161, 173-182 (2002).

14. Krause, D. S. et al. Multi-organ, multi-lineage engraftment by a single bone marrow-derived stem cell. Cell 105, 369-377 (2001).

15. VanWinkle, L. S. et al. Maintenance of differentiated murine Clara cells in microdissected airway cultures. Am. J. Respir. Cell Mol. Biol. 14, 586-598 (1996).

16. Devereux, T. R. and Fouts, J. R. Isolation and identification of Clara cells from rabbit lung. In vitro. 16, 958-968 (1980).

17. Fuchs, S. et al. Differentiation of human alveolar epithelial cells in primary culture: morphological characterization and synthesis of caveolin-1 and surfactant protein-C. Cell Tissue Res. 311, 31-45 (2003).

18. Nielsen, N. et al. Aquaporins in complex tissues. II. Subcellular distribution in respiratory and glandular tissues of the rat. Am. J. Physiol. 273, C1549-C1561 (1997).

19. Chen, J. H. et al. Plasma proteome of severe acute respiratory syndrome analyzed by two-dimensional gel electrophoresis and mass spectrometry Proc. Natl. Acad. Sci. U. S. A. 101, 17039-17044 (2004).

20. Ng, M. L. at al. Proliferative growth of SARS coronavirus in Vero E6 cells. J. Geneal Viol. 84, 3291-3303 (2003).

21. Albert, M. R. et al. Murine epidermal label-retaining cells isolated by flow cytometry do not express the stem cell markers CD34, Sca-1, or Flk-1. J. Invest Dermatol. 117, 943-948 (2001).

22. Meyts E. R. et al. Developmental expression of POU5F1 (Oct-3/4) in normal and dysgenetic human gonads. Human Reprod. 19, 1338-1344. (2004)

23. Shimozaki, J. K. et al. Involvement of Oct3/4 in the enhancement of neuronal differentiation of ES cells in neurogenesis-inducing cultures. Development 130, 2505-2512 (2003).

24. Bonyadi, M. et al. Mesenchymal progenitor self-renewal deficiency leads to age-dependent osteoporosis in Sca-1/Ly-6A null mice. Proc. Natl. Acad. Sci. U.S.A. 95, 9471-9476 (1998).

25. Yeh, S. H. et al. Characterization of severe acute respiratory syndrome coronavirus genomes in Taiwan: molecular epidemiology and genome evolution. Proc. Natl. Aced. Sci. U.S.A. 101, 2542-2547 (2004).

26. Burleson, F. G., Chambers, T. M. & Wiedbrauk, D. L. Plaque assay. in Virology: A Laboratory Manual. (eds Burleson, F. G., Chambers, T. M. & Wiedbrauk, D. L.) 74 84 (Academic Press, San Diego, 1992)

27. Ito, T. and Kawaoka, Y. Host-range barrier of influenza A viruses. Vet. Microbiol. 74, 71-75 (2000).

1. Isolated pulmonary stem cells, the isolated pulmonary stem cells being slowly dividing cells forming individual colonies in vitro, the pulmonary stem cells expressing the marker Oct-4, the pulmonary stem cells able to undergoing terminal differentiation into a mature phenotype.

2. The isolated pulmonary stem cells of claim 1, wherein the pulmonary stem cells further express the markers SSEA-I and Sca-1.

3. The isolated pulmonary stem cells of claim 1, wherein the pulmonary stem cells further express the markers SSEA-3 SSEA-4 and Sca-1.

4. A method to identify stem cells of a tissue, the method comprising:
identifying slowly dividing Oct-4 expressing cells of the tissue,
the identified slowly dividing Oct-4 expressing cells able to form colonies in vitro, the identified slowly dividing Oct-4 expressing cells, upon isolation in primary culture, able to differentiate in a mature phenotype.

5. The method of claim 4, wherein identifying slowly dividing Oct-4 expressing cells comprises
identifying the slowly dividing cells of the tissue; and
identifying the slowly dividing cells expressing the stem cell marker Oct-4.

6. The method of claim 4, wherein identifying slowly dividing cells of the tissue is performed by identifying BrdU retaining cells of the tissue.

7. The method of claim 4, wherein identifying the Oct-4 expressing cells of the tissue is performed by immunofluorescence, RT-PCR or quantitative PCR.

8. A kit of parts for the identification of stem cell of a tissue, the kit of parts comprising
a first identifier for the identification of slowly dividing cells; and
a second identifier for the identification of the expression of the marker Oct-4,
the first and the second identifier to be used to identify slowly dividing Oct-4 expressing cells, the identified slowly dividing Oct-4 expressing cells able to form colonies in vitro and to differentiate in a mature phenotype, being stem cells.

9. The kit of parts of claim 8, wherein the first identifier is BrdU and the second identifier is selected from the group consisting of primary and secondary antibodies and oligonucleotides.

10. The kit of parts of claim 8, wherein the tissue is a murine tissue and the kit further comprises a third identifier for the identification of an additional marker, the additional marker selected from the group consisting of SSEA-1 and SCA-1.

11. The kit of parts of claim 8, wherein the tissue is a human tissue and the kit further comprises a third identifier for the identification of an additional marker, the additional marker selected from the group consisting of SSEA-3, SEEA-4 and Sca-1.

12. The kit of parts of claim 8, further comprising a fourth identifier for the identification of the mature phenotype.

13. A method for cultivating adult stem cells in vitro, the method comprising:
providing a tissue comprising tissue cells;
isolating the tissue cells from the tissue;
incubating the isolated tissue cells in a suitable medium;
applying to the isolated incubated cell a cell sorting procedure to obtain colonies in primary cultures; and
selecting the colonies in primary colonies that express a marker Oct-4, the colonies able to differentiate into mature type.

14. The method of claim 13, wherein the adult stem cells are pulmonary stem cells.

15. A method for identifying an infective agent able to infect pulmonary cells, the method comprising
contacting an infective agent with an isolated pulmonary stem cell;
detecting the level of infection of the isolated pulmonary stem cell by the infective agent; and
comparing the detected level of infection with a predetermined threshold level, the threshold level indicative of development of infection of the cell.

16. A method for the production of a virus able to infect pulmonary cells, the method comprising:
contacting a virus particle with an isolated pulmonary stem cell in a pulmonary stem cell culture; and
collecting virus particles from the cell culture.

17. The method of claim 16, wherein contacting a virus particle with an isolated pulmonary stem cell in a culture is able be performed in a serum-free culture condition.

18. The method of claim 17, wherein the viral particle is selected from the group consisting of influenza and avian flu viral particles.

19. The method of claim 16, wherein the viral particle is a SARS viral particle.

20. A method for identifying a compound interfering with infection of a pulmonary stem cell by an infective agent, the method comprising
contacting a candidate compound with an isolated pulmonary stem cell;
contacting the isolated pulmonary stem cell with a candidate infective agent;
detecting the level of infection of the isolated pulmonary stem cell by the infective agent; and
comparing the detected level of infection with a predetermined threshold level, the threshold level indicative of development of infection of the cell.

21. The method of claim 20, wherein the infective agent is a virus, the virus selected from the group consisting of influenza and avian flues viruses.

22. The method of claim 20, wherein the infective agent is SARS virus.
